(19) United States
(12) Patent Application Publication
(10) Pub. No.: US 2012/0021481 A1
(43) Pub. Date: Jan. 26, 2012

(54) ELECTROMECHANICAL LYSING OF ALGAE CELLS

(75) Inventors: Robert E. Hebner, Austin, TX (US); Kent Davey, Edgewater, FL (US); Michael D. Werst, Manor, TX (US); Rhykka Connelly, Austin, TX (US)

(73) Assignee: BOARD OF REGENTS, THE UNIVERSITY OF TEXAS SYSTEM, Austin, TX (US)

(21) Appl. No.: 13/186,282

(22) Filed: Jul. 19, 2011

Related U.S. Application Data
(60) Provisional application No. 61/365,973, filed on Jul. 20, 2010.

(51) Int. Cl. C12N 13/00; C07K 1/14; C10L 1/18; C07H 1/06; C11C 3/00; C12M 1/42; C11B 1/00

(52) U.S. Cl. 435/173.7; 435/306.1; 435/289.1; 530/412; 554/175; 536/127; 554/161; 44/385

(57) ABSTRACT

Methods and electroporation devices for electrical treatment of algal cell cultures for release of lipids and proteins are described herein. The method of the present invention exploits the differences in electrical time constants for the media inside the cell and outside the cell to produce a net force to cause cellular lysis and extract cellular components. The method of the present invention can be used in the treatment of flocculated as well as unflocculated algal cell cultures. The device of the present invention provides efficient cell lysing in a low-energy cost set-up.

### CROSS-REFERENCE TO RELATED APPLICATIONS

This patent application claims priority to U.S. Provisional Patent Application No. 61/365,973 filed on Jul. 20, 2010 and entitled “Electromechanical Lysing of Algae Cells”, which is hereby incorporated by reference in its entirety.

### TECHNICAL FIELD OF THE INVENTION

The present invention relates in general to the electromechanical manipulation of biological cells, primarily, but not exclusively, for the purpose of extracting chemical compounds from the interior of the cells, and more particularly to an electromechanical process for the breaching or removal of an algal cell wall.

### STATEMENT OF FEDERALLY FUNDED RESEARCH

None.

### REFERENCE TO A SEQUENCE LISTING

None.

### BACKGROUND OF THE INVENTION

Without limiting the scope of the invention, its background is described in connection with methods for extraction chemicals from inside of algae/biological cells involved mechanical and/or chemical disruption of the cell wall.

U.S. Patent Publication No. 20080220491, Zimmermann et al. 2008 (hereinafter Zimmermann) relates to methods for electrical treatment of biological cells, in particular for electroporation or electropermeabilisation of biological cells which are arranged on a fixed carrier element, as well as electroporation devices for carrying out such methods. The Zimmermann invention describes methods for electrical treatment of biological cells, in particular using electrical field pulses, involving the steps: arrangement of the cells on apertures of a solid planar carrier element (3) which divides a measuring chamber into two compartments; and temporary formation of an electrical treatment field which permeates the cells, wherein an alternating-current impedance measurement takes place on the carrier element, and from the result of the alternating-current impedance measurement, a degree of coverage of the carrier element and/or healing of the cells after electrical treatment are/is acquired. The invention also describes devices for implementing the methods.

U.S. Patent Publication No. 20090061504 (Davey, 2009) discloses an apparatus for performing magnetic electroporation. The required electric field for electroporation in the Davey invention is generated using a pulsed magnetic field through a closed magnetic yoke, such as a toroid, placed in a flow path of a fluid medium to be processed. The fluid medium flows through the orifice of the magnetic yoke, with the fluid medium flowing through and around the yoke. The required power to send a maximum flux through the magnetic yoke is less than the required power in a conventional apparatus for performing electroporation.

U.S. Patent Publication No. 20090087900 (Davey and Hebner, 2009) describes two apparatuses capable of performing electroporation. The first apparatus uses a Marx generator with a substantial change from its original waveform. The second apparatus does not use a Marx generator.

### SUMMARY OF THE INVENTION

The approaches heretofore used for extraction of chemicals from inside of algae cells involved mechanical and/or chemical disruption of the cell wall. These approaches involved drying, grinding, and chemical extraction; slowly increasing and suddenly decreasing external pressure so that the cell explodes; or by applying short wavelength pressure waves such as those produced by bubble collapse during ultrasonic excitation. The present invention is an electromechanical process to open the cell. The invention exploits the fact that the electrical time constants can be sufficiently different for the media inside the cell and outside the cell. In equilibrium, the electric charge distribution inside of the cell compensates for any external charge distribution induced by an imposed electric field. The same is not true under transient conditions, however. Because of the inherent differences between electrical time constants inside and outside the cell, a net force can be produced.

In one embodiment the present invention provides a method for electrical treatment of one or more biological cells comprising the steps of: (i) providing the one or more biological cells suspended or surrounded by a lysing medium comprising a fresh water, a salt water, a brackish water, a growth medium, a culture medium or combinations thereof, wherein an electrical conductivity of the lysing medium is different from the electrical conductivity of a cell membrane and the cytoplasm of the one or more biological cells, (ii) applying a time varying electromagnetic field to the one or more biological cells using one or more electrode pairs placed in the lysing medium or external to the lysing medium, wherein the applied electromagnetic field results in a mechanical force on a cell membrane comprising a force stress, and (iii) applying and rapidly switching off one or more voltage pulses to the one or more biological cells resulting in a reversal in the direction of the force stress causing a lysis of the one or more biological cells.

The electrical treatment method described hereinabove further comprising the steps of: releasing one or more cellular components from the lysed biological cells into the lysing medium and separating and collecting the released cellular components for further processing. In one aspect the cellular components that are released comprise neutral lipids, proteins, triglycerides, sugars or combinations and modifications thereof. In another aspect the neutral lipids, triglycerides or both are converted to yield a fatty acid methyl ester (FAME), a biodiesel or a biofuel. The one or more biological cells described in the method of the instant invention comprise algal cells, bacterial cells, viral cells or combinations thereof

The algal cells described in the method hereinabove are selected from a division comprising Chlorophyta, Cyanophyta (Cyanobacteria), Rhodophyta (red algae), and Heterokontophyt. In one aspect the one or more algal cells comprise microalgae selected from a class comprising Bacillariophyceae, Eustigmatophyceae, and Chrysophyceae. In another aspect the microalgal genera are selected from the group consisting of Nannochloropsis, Chlorella, Dunaliella, Scenedesmus, Selenastrum, Oscillatoria, Phormidium, Spirulina, Amphora, and Ochromonas. In yet another aspect the microalgal species are selected from the group consisting of Achnanthes orientalis, Agmenellum spp., Amphiprora hyaline, Amphoracoffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorellakessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Effipsoidon sp., Euglena spp., Franceia sp., Fragilaria crotonensis, Fragilaria sp., Gleocapsa sp., Gloeothamnion sp., Haematococcus pluvialis, Hymenomonas sp., lsochrysis aff galbana, lsochrysis galbana, Lepocinclis, Micractinium, Micractinium, Monoraphidium minutum, Monoraphidium sp., Nannochloris sp., Nannochloropsissalina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrine, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheriaacidophila, Pavlova sp., Phaeodactylum tricomutum, Phagus, Phormidium, Platymonas sp., Pleurochrysis camerae, Pleurochrysis dentate, Pleurochrysis sp., Prototheca wickerhamii, Prototheca stagnora, Prototheca portoricensis,Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

In yet another aspect the electrical treatment is carried out in a batch or a continuous processing mode. In a specific aspect the strength of the applied electromagnetic field ranges from 0.5 kV/cm to 500 kV/cm and the electromagnetic field is applied for a time duration ranging from a tenth of a microsecond to a few tens of microseconds.

In another embodiment the instant invention discloses a method for lysing and releasing one or more cellular components comprising neutral lipids, proteins, triglycerides, sugars or combinations and modifications thereof by electroporation of one or more algal cell membranes comprising the steps of: providing the one or more algal cells suspended or surrounded by a lysing medium comprising fresh water, salt water, brackish water, a growth medium, a culture medium or combinations thereof, wherein an electrical conductivity of the lysing medium is different from the electrical conductivity of the cell membrane and of a cytoplasm of the one or more algal cells, applying a time varying electromagnetic field to the algal cells using one or more electrode pairs placed in the lysing medium or external to the lysing medium, wherein the electromagnetic field applies a mechanical force comprising a force stress on an algal cell membrane, applying and rapidly switching off one or more constant amplitude voltage pulses to the one or more algal cells resulting in a reversal in the direction of the radial force stress followed by an expansion of the cells in the radial direction causing a lysis of the algal cells, and lysing the one or more algal cells to release one or more cellular components into the lysing medium. The method as described herein further comprises the steps of separating and collecting the neutral lipids, the triglycerides or both from the released cellular components for further processing and converting the neutral lipids, the triglycerides or both to yield a FAME, a biodiesel or a biofuel.

In a related aspect to the lysis method disclosed herein the algal cells comprise microalgae or macroalgae selected from the group consisting of diatoms (bacillariophytes), green algae (chlorophytes), blue-green algae (cyanophytes), golden-brown algae (chrysophytes), haptophytes, freshwater algae, saltwater algae, Amphipleura, Amphora, Chaetoceros, Cyclotella, Cymbella, Fragilaria, Hantzschia, Navicula, Nitzschia, Phaeodactylum, Thalassiosira Ankistrodesmus, Botryococcus, Chlorella, Chlorococcum, Dunaliella, Monoraphidium, Oocystis, Scenedesmus, Nannochloropsis, Tetraselmis, Chlorella, Dunaliella, Oscillatoria, Synechococcus, Boekelovia, Isochysis, and Pleurochysis. In a specific aspect the algae is Chlorella or Nannochloropsis. In other aspects related to the method of the instant invention the cell density of the one or more algal cells ranges from a single cell to a largest cell density, wherein an external electrical conductivity is determined by the lysing medium. The strength of the applied electromagnetic field for lysis ranges from 0.5 kV/cm to 500 kV/cm and the said field is applied for a time duration ranging from a tenth of a microsecond to a few tenths of a microsecond and the step of lysing is carried out in a batch or a continuous processing mode.

Yet another embodiment is related to a method for lysing a flocculated or unflocculated algal cell culture to release one or more cellular components comprising neutral lipids, proteins, triglycerides, sugars or combinations and modifications thereof by electroporation of an algal cell membrane comprising the steps of: (i) providing the one or more flocculated or unflocculated algal cell cultures suspended or surrounded by a lysing medium which may be a fresh water, a salt water, a brackish water, a growth medium, a culture medium or combinations thereof, wherein an electrical conductivity of the lysing medium is different from the electrical conductivity of the cell membrane of the one or more algal cells, (ii) applying multiple pulses of a time varying electromagnetic field to the flocculated or unflocculated algal cells using one or more electrode pairs placed in the lysing medium or external to the lysing medium, wherein the electromagnetic field applies a mechanical force comprising a radial force stress compressing the cells inward along a radial direction of the applied electromagnetic field and an axial force stress elongating the cells in a direction along an axis of the applied electromagnetic field, (iii) applying and rapidly switching off one or more constant amplitude voltage pulses to the flocculated or unflocculated algal cells, (iv) inducing a reversal in the direction of the radial force stress followed by an expansion of the cells in the radial direction causing a lysis of the algal cells, and (iv) lysing the one or more algal cells to release one or more cellular components into the lysing medium.

The lysing method of the instant invention further comprises the steps of: separating and collecting the neutral lipids, the triglycerides or both from the released cellular components for further processing and converting the neutral lipids, the triglycerides or both to yield a FAME, a biodiesel or a biofuel.

The algal cells undergoing the lysing step comprise microalgae or macroalgae selected from the group consisting of diatoms (bacillariophytes), green algae (chlorophytes), blue-green algae (cyanophytes), golden-brown algae (chrysophytes), haptophytes, freshwater algae, saltwater algae, Amphipleura, Amphora, Chaetoceros, Cyclotella, Cymbella, Fragilaria, Hantzschia, Navicula, Nitzschia, Phaeodactylum, Thalassiosira Ankistrodesmus, Botryococcus, Chlorella, Chlorococcum, Dunaliella, Monoraphidium, Oocystis, Scenedesmus, Nannochloropsis, Tetraselmis, Chlorella, Dunaliella, Oscillatoria, Synechococcus, Boekelovia, Isochysis, and Pleurochysis.

The present invention further describes a system for producing a biodiesel, a FAME, a biofuel or combinations and modifications thereof from an algal cell culture comprising: (i) an algal growth tank or a cultivation tank for growing the one or more algal species in a presence of water and other growth factors selected from the group consisting of nutrients, minerals, CO₂, air, and light, (ii) a harvesting vessel for harvesting the cultivated algae from the growth tank, wherein the algae are harvested by one or more methods selected from the group consisting of centrifugation, autoflocculation, chemical flocculation, froth flotation and ultrasound, (iii) a concentration tank wherein the harvested algae is dewatered to concentrate the algae, (iv) a lysis tank comprising a lysing medium for electromechanically lysing the concentrated algae to release one or more cellular components comprising neutral lipids, proteins, triglycerides, sugars or combinations and modifications thereof, wherein an electrical conductivity of the lysing medium is different from the electrical conductivity of an algal cell membrane, wherein the lysing is accomplished by an electroporation device comprising: (a) single or multiple pairs of electrodes for applying a single pulse or multiple pulses of a time varying electromagnetic field to the algal cells, wherein the electromagnetic field applies a mechanical force on the algal cell membrane comprising a radial force stress compressing the cells inward along a radial direction of the applied electromagnetic field and an axial force stress elongating the cells in a direction along an axis of the applied electromagnetic field and (b) an apparatus for applying and rapidly switching off one or more constant amplitude voltage pulses to the algal cells resulting in a reversal in the direction of the radial force stress followed by an expansion of the cells in the radial direction causing a lysis of the algal cells, (v) a separation vessel for separating the released algal lipids and triglycerides from the lysing medium and other released cellular components, and (vi) a reaction vessel for converting the separated algal lipids, triglycerides to a biodiesel, a FAME, a biofuel or combinations or modifications thereof by a transesterification reaction.

The algal species that are processed in the system described hereinabove comprise microalgae or macroalgae selected from the group consisting of diatoms (bacillariophytes), green algae (chlorophytes), blue-green algae (cyanophytes), golden-brown algae (chrysophytes), haptophytes, freshwater algae, saltwater algae, Amphipleura, Amphora, Chaetoceros, Cyclotella, Cymbella, Fragilaria, Hantzschia, Navicula, Nitzschia, Phaeodactylum, Thalassiosira Ankistrodesmus, Botryococcus, Chlorella, Chlorococcum, Dunaliella,

Monoraphidium, Oocystis, Scenedesmus, Nannochloropsis, Tetraselmis, Chlorella, Dunaliella, Oscillatoria, Synechococcus, Boekelovia, Isochysis, and Pleurochysis.

The present invention in one embodiment discloses a device for electrical treatment of biological cells comprising: a chamber or a vessel comprising flocculated or unflocculated biological cells suspended or surrounded by a lysing medium which may be fresh water, salt water, brackish water, a growth medium, a culture medium or combinations thereof, wherein an electrical conductivity of the lysing medium is different from the electrical conductivity of a cell membrane of the one or more biological cells, one or more pairs of electrodes for applying single or multiple pulses of a time varying electromagnetic field to the biological cells, wherein the applied electromagnetic field results in a mechanical force on the cell membrane comprising a radial force stress compressing the cells inward along a radial direction of the applied electromagnetic field and an axial force stress elongating the cells in a direction along an axis of the applied electromagnetic field, an apparatus for applying and rapidly switching off one or more constant amplitude voltage pulses to the biological cells resulting in a reversal in the direction of the radial force stress followed by an expansion of the cells in the radial direction causing a lysis of the algal cells, and one or more optional collecting vessels, receivers, separators or combinations for processing the released cellular components.

In one aspect of the device the electrodes are profiled to create an uniform field and minimal voltage stress concentration. In another aspect the cellular components comprise neutral lipids, proteins, triglycerides, sugars or combinations and modifications thereof. In another aspect the neutral lipids, triglycerides or both are converted to yield a FAME, a biodiesel or a biofuel. In yet another aspect the one or more biological cells comprise algal cells, bacterial cells, viral cells or combinations thereof.

The algal cells described hereinabove are selected from a division comprising Chlorophyta, Cyanophyta (Cyanobacteria), Rhodophyta (red algae), and Heterokontophyt. In one aspect the one or more algal cells comprise microalgae selected from a class comprising Bacillariophyceae, Eustigmatophyceae, and Chrysophyceae. In another aspect the microalgal genera are selected from the group consisting of Nannochloropsis, Chlorella, Dunaliella, Scenedesmus, Selenastrum, Oscillatoria, Phormidium, Spirulina, Amphora, and Ochromonas. In yet another aspect the microalgal species are selected from the group consisting of Achnanthes orientalis, Agmenellum spp., Amphiprora hyaline, Amphoracoffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorellakessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Effipsoidon sp., Euglena spp., Franceia sp., Fragilaria crotonensis, Fragilaria sp., Gleocapsa sp., Gloeothamnion sp., Haematococcus pluvialis, Hymenomonas sp., lsochrysis aff. galbana, lsochrysis galbana, Lepocinclis, Micractinium, Micractinium, Monoraphidium minutum, Monoraphidium sp., Nannochloris sp., Nannochloropsissalina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrine, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheriaacidophila, Pavlova sp., Phaeodactylum tricomutum, Phagus, Phormidium, Platymonas sp., Pleurochrysis carterae, Pleurochrysis dentate, Pleurochrysis sp., Prototheca wickerhamii, Prototheca stagnora, Prototheca portoricensis,Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

In other aspects the electrical treatment is carried out in a batch or a continuous processing mode and the strength of the applied electromagnetic field ranges from 0.5 kV/cm to 500 kV/cm. In a related aspect the electromagnetic field is applied for a time duration ranging from a tenth of a microsecond to a few tens of microseconds.

The present invention also includes a device for electrical treatment for a release of one or more cellular components comprising neutral lipids, proteins, triglycerides, sugars or combinations and modifications thereof from one or more flocculated or unflocculated algal cell cultures comprising: (i) a chamber or a vessel comprising flocculated or unflocculated algal cells suspended or surrounded by a lysing medium which may be a fresh water, a salt water, a brackish water, a growth medium, a culture medium or combinations thereof, wherein an electrical conductivity of the lysing medium is different from the electrical conductivity of a cell membrane of the one or more algal cells, (ii) one or more pairs of electrodes for applying single or multiple pulses of a time varying electromagnetic field to the algal cells, wherein the applied electromagnetic field results in a mechanical force on the cell membrane comprising a radial force stress compressing the cells inward along a radial direction of the applied electromagnetic field and an axial force stress elongating the cells in a direction along an axis of the applied electromagnetic field, (iii) an apparatus for applying and rapidly switching off one or more voltage pulses to the algal cells resulting in a radial force stress followed by an expansion of the cells causing a lysis of the algal cells, and (iv) one or more optional collecting vessels, receivers, separators or combinations for processing the released cellular components. In one aspect the neutral lipids, the triglycerides or both are converted to yield a FAME, a biodiesel or a biofuel. In another aspect the algal cells comprise microalgae or macroalgae selected from the group consisting of diatoms (bacillariophytes), green algae (chlorophytes), blue-green algae (cyanophytes), golden-brown algae (chrysophytes), haptophytes, freshwater algae, saltwater algae, Amphipleura, Amphora, Chaetoceros, Cyclotella, Cymbella, Fragilaria, Hantzschia, Navicula, Nitzschia, Phaeodactylum, Thalassiosira Ankistrodesmus, Botryococcus, Chlorella, Chlorococcum, Dunaliella, Monoraphidium, Oocystis, Scenedesmus, Nannochloropsis, Tetraselmis, Chlorella, Dunaliella, Oscillatoria, Synechococcus, Boekelovia, Isochysis, and Pleurochysis. In other aspects the algae is Chlorella or Nannochloropsis.

### BRIEF DESCRIPTION OF THE DRAWINGS

For a more complete understanding of the features and advantages of the present invention, reference is now made to the detailed description of the invention along with the accompanying figures and in which:

FIG. 1 is a schematic illustration of a system for processing algae for the extraction of a biodiesel or a biofuel according to an embodiment of the present invention;

FIG. 2 is a schematic illustration of an algal model and coordinate system;

FIG. 3 is a schematic showing charge generation at algal membrane interfaces

FIG. 4 is a plot showing the applied voltage pulse;

FIG. 5 is a plot showing the forces on the algal cell membrane;

FIG. 6 is a simulation plot of a radial compression force;

FIG. 7 is a simulation plot of an axial compression force;

FIG. 8 is a plot showing a short applied voltage pulse;

FIG. 9 is a plot showing a radial force reversal;

FIG. 10 is a plot showing rapid voltage reversal;

FIG. 11 is a plot showing a large force reversal;

FIG. 12 is a histogram showing Chlorella protein release as an indicator of lysis efficiency;

FIGS. 13A and 13B are histogram plots showing neutral lipid release as an indicator of lysis efficiency in Chlorella detected using: (FIG. 13A): Nile Red and (FIG. 13B) BODIPY 493;

FIGS. 14A and 14B are histogram plots showing neutral lipid release as an indicator of lysis efficiency in Nannochloropsis detected using: (FIG. 14A): Nile Red and (FIG. 14B) BODIPY 493;

FIGS. 15A and 15B are scanning electron microscope photographs of sample of Scenedesmus, a specific type of algae, before (FIG. 15A) and after (FIG. 15B) electromechanical lysing; and

FIGS. 16A and 16B are scanning electron microscope photographs of samples of Chlorella, a specific type of algae, before (FIG. 16A) and after (FIG. 16B) electromechanical lysing.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below.

Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term “algae” represents a large, heterogeneous group of primitive photosynthetic organisms which occur throughout all types of aquatic habitats and moist terrestrial environments. Nadakavukaren et al., Botany. An Introduction to Plant Biology, 324-325, (1985). The term “algae” as described herein is intended to include the species selected from the group consisting of the diatoms (bacillariophytes), green algae (chlorophytes), blue-green algae (cyanophytes), golden-brown algae (chrysophytes), haptophytes, freshwater algae, saltwater algae, Amphipleura, Amphora, Chaetoceros, Cyclotella, Cymbella, Fragilaria, Hantzschia, Navicula, Nitzschia, Phaeodactylum, Thalassiosira Ankistrodesmus, Botryococcus, Chlorella, Chlorococcum, Dunaliella, Monoraphidium, Oocystis, Scenedesmus, Nanochloropsis, Tetraselmis, Chlorella, Dunaliella, Oscillatoria, Synechococcus, Boekelovia, Isochysis and Pleurochysis. The term also includes microalgae selected from a class comprising Bacillariophyceae, Eustigmatophyceae, and Chrysophyceae and genera are selected from the group consisting of Nannochloropsis, Chlorella, Dunaliella, Scenedesmus, Selenastrum, Oscillatoria, Phormidium, Spirulina, Amphora, and Ochromonas. The microalgal species may be selected from the group consisting of Achnanthes orientalis, Agmenellum spp., Amphiprora hyaline, Amphoracoffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorellakessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Effipsoidon sp., Euglena spp., Franceia sp., Fragilaria crotonensis, Fragilaria sp., Gleocapsa sp., Gloeothamnion sp., Haematococcus pluvialis, Hymenomonas sp., lsochrysis aff galbana, lsochrysis galbana, Lepocinclis, Micractinium, Micractinium, Monoraphidium minutum, Monoraphidium sp., Nannochloris sp., Nannochloropsissalina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrine, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheriaacidophila, Pavlova sp., Phaeodactylum tricomutum, Phagus, Phormidium, Platymonas sp., Pleurochrysis camerae, Pleurochrysis dentate, Pleurochrysis sp., Prototheca wickerhamii, Prototheca stagnora, Prototheca portoricensis,Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

The term “electromechanical” as used herein refers to a mechanical vibration, flexing or oscillation in response to an energetic stimulus. Examples of such energetic stimulus include, without limitation, applied electric and magnetic fields. The term “lysing” refers to the action of rupturing the cell wall and/or cell membrane of a cell. The term “lysing” does not require that the cells be completely ruptured; rather, “lysing” can also refer to the release of intracellular material.

The term “interface” as used herein indicates a boundary between any two immiscible phases. The term “homogenizer” is used in the general sense of a grinder, and often no pressure limitations or initial, i.e., prehomogenization, particle size required in order to achieve the desired particle size are specified. The term “protein” refers to a macromolecule comprising one or more polypeptide chains. A “polypeptide” is a polymer of amino acid residues joined by peptide bonds, whether produced naturally or synthetically. Polypeptides of less than about 10 amino acid residues are commonly referred to as “peptides.” A protein may also comprise non-peptidic components, such as carbohydrate groups. Carbohydrates and other non-peptidic substituent's may be added to a protein by the cell in which the protein is produced, and will vary with the type of cell. Proteins are defined herein in terms of their amino acid backbone structures; substituent's such as carbohydrate groups are generally not specified, but may be present nonetheless.

The present invention describes methods and devices for extracting valuable cellular components from algal and other biological cells by electromechanical manipulation of the differences in electrical time constants of the media inside and outside of the cell. The electromechanical lysing method of the instant invention yields refinery-ready oil and biomass bioproducts that is scalable and transportable.

Algae are among the most promising next-generation sources for biofuels. They grow quickly, use solar energy efficiently, capture and reuse CO₂, and do not compete with the food supply. Algae yields 2,000-15,000 gallons of fuel per acre, compared with 50 gallons for soybean oil and 650 gallons for palm oil.

Although there is a great potential for the use of algae as a source of biofuels a number of technological developments are needed before recovery of oil will be economical. Key issues deal with the large amounts of water involved in growing algae which typically grows to concentrations of less than one percent. Harvesting and dewatering algae from low-density cultures has been achieved but this often yields a paste whose physical properties make subsequent processing difficult. For example, these pastes still contain considerable amounts of water that prevent direct mixing with organic solvents and they do not flow through extraction equipment. Traditional methods for extracting oil from seeds are generally ineffective at the size scale of algae cells. Instead, extracting oil from algae typically involves drying the algae, breaking down the cell walls with a solvent, then removing the solvent and biomass to leave behind the oil. Methods such as supercritical extraction are uneconomical for commodity products such as fuel. Solvent extraction requires distillation of an extract to separate the solvent from the oil. Also, a steam stripper is usually required to recover the residual solvent dissolved or entrained within the exiting algal concentrate. The solvent extraction technique requires contactor equipments or phase separation equipments, a distillation system and a steam stripper along with varying heat exchangers, surge tanks and pumps. Also steam and cooling water are required. Because these methods require large amounts of energy, large volumes of water, and chemical solvents, they are ultimately too expensive and too environmentally unsound to be viable for large-scale fuel production. Thus, extracting the oil from the algae cost-effectively is a significant challenge.

Electroporation of biological cells to generate transitory pores in the cell membrane by exposure to high-voltage electric potentials has been previously described. U.S. Patent Publication No. 20090061504 (Davey, 2009), incorporated herein by reference, describes an apparatus and a method for performing magnetic electroporation to allow influx or efflux of large molecules from a biological cell, including algal cells. The apparatus of the Davey invention comprises a ferrous toroid placed within a fluid chamber and a fluid medium flowing through the chamber such that the fluid medium flows around the ferrous toroid. Furthermore, the electric field has a closed path within the fluid medium around the ferrous toroid.

Davey and Hebner (2009) in U.S. Patent Publication No. 20090087900 (incorporated herein by reference) disclose electromechanical manipulation of algal cells to cause electrodistention and subsequent lysis. The two apparatuses capable of causing electrodistention of the algal cells as described in the Davey and Hebner invention comprise a Marx generator and a cable pulse device. The electromechanical manipulation by the device described in the 20090087900 publication leads to tearing, stretching, and/or puncture of the cells. The large scale cell wall destruction can be visually observed and also be inferred in the degree of lipid produced.

This invention is an electromechanical process to open the cell and extracting the oil from the algae by breaking down cell walls using electromagnetic forces, thereby eliminating energy-consuming drying stages and the use of chemical solvents. The low-energy method of the instant invention works well in dilute concentrations, and higher concentrations yield oil even more efficiently. The present invention exploits the fact that the electrical time constants can be sufficiently different for the media inside the cell and outside the cell. In equilibrium, the electric charge distribution inside of the cell compensates for any external charge distribution induced by an imposed electric field. The same is not true under transient conditions, however. Because of the inherent differences between electrical time constants inside and outside the cell, a net force can be produced.

The present invention for electromechanical lysis offers significant advantages over existing devices and the prior art. The low-energy operation of the set-up of the present invention works well in dilute concentrations. The device of the present invention can be adapted for use in releasing cellular components from one or more flocculated or unflocculated algal cell cultures. The device described herein in various embodiments may be placed within a lysing chamber or may be external to the chamber. The method promotes efficient lysing of the algal cells by permitting a very rapid force application caused by the application and switching off of one or more voltage pulses to the flocculated algal cells. This resulting in a reversal in the direction of the radial force stress on the algal cells followed by an expansion of the cells in the radial direction causing a lysis of the algal cells.

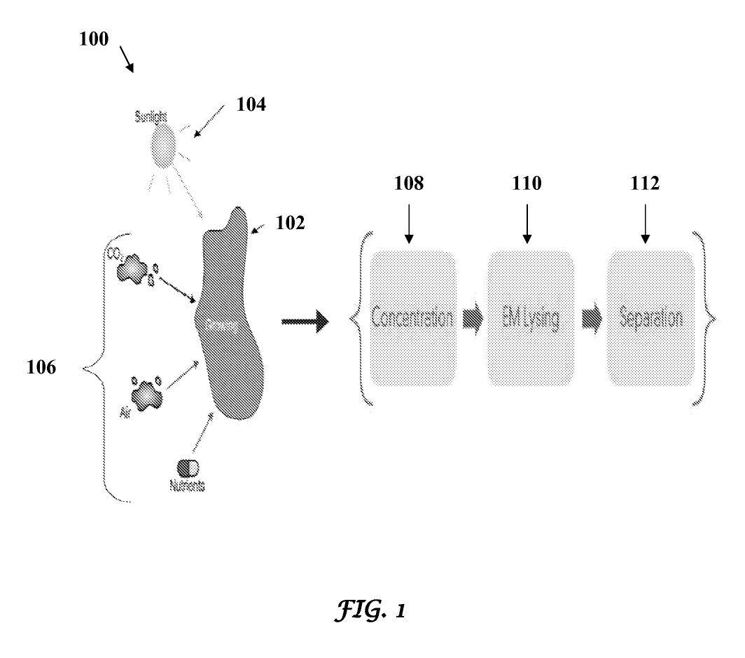

FIG. 1 is a schematic illustration of a typical system 100 according to an embodiment of the instant invention. The system 100 comprises a cultivation tank or a pond (as shown in FIG. 1) 102. The algae grow in the presence of sunlight 104 or artificial light in the presence of nutrients 106 (selected from air, CO₂, and other nutrients). After growth the algae are harvested and concentrated in step 108, wherein the algae is dewatered, and the water is returned to the pond 102. Step 108 prepares the algae for further processing in the most cost effective manner. The concentration step 108 is followed by an electromechanical (EM) lysing step 110 of the instant invention that uses very little energy to destroy the algal cell walls quickly, thereby releasing the oil from the algae for maximum recovery. In the final separation step 112, the oil is separated from the lysing medium and other released cellular components by physical or chemical separation methods. The separated algal oils are then processed further for conversion to biodiesel, biofuels or other valuable commodities.

The methodology of the present invention maximizes valuable product recovery from algae: algal oil, and biomass that can be used as feedstock, fertilizer, or fuel. Because the system described herein avoids chemical solvents other systems rely upon, the byproducts, water and biomass are valuable. Once the oil is removed, the water can be returned to the cultivation system and the remaining biomass can be used as edible or combustible material.

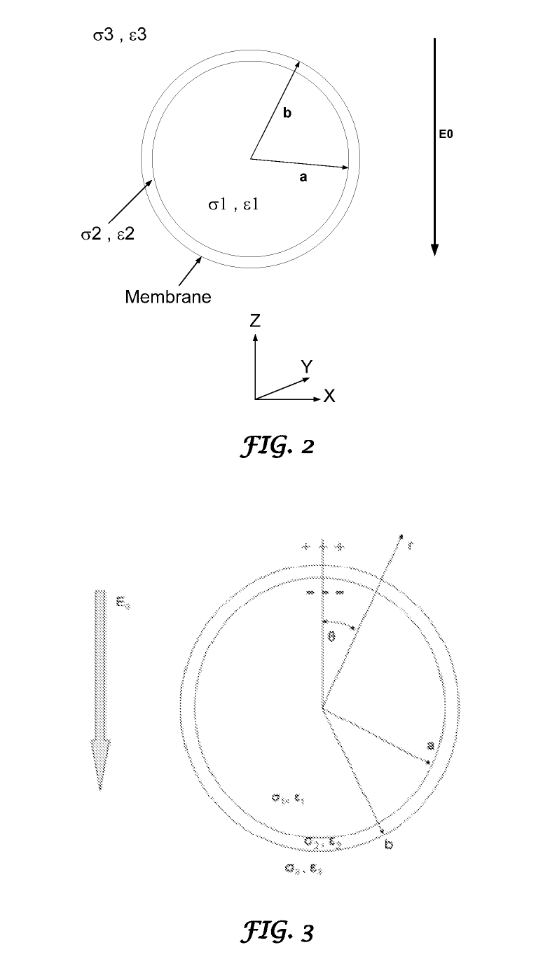

Specifically, a simple algae cell can be represented schematically as shown in FIG. 2. The alga is assumed spherical with a thin membrane separating it from ambient water. The process works as well or better for non-spherical algae cells. For clarity, consider the simplest situation in which, at distances far from the cell, the applied electric field (time dependent) is directed along a single axis. In the numerical simulation as in practice, this is realized by placing the alga between two large electrode surfaces. The numerical boundary condition is that at large radial distances from the alga the electric field is purely axial.

The claimed behavior can be simulated using conventional computational tools. The simulation assumes axial symmetry for computational convenience. Thus, the solutions obtained are fully three dimensional.

The electric potential (voltage) applied between the two electrodes is a function of time. The simulation solves for the quasi-static electric potential distribution throughout the entire space of the problem. In this approximation, the magnetic field produced by current flow is small enough to be ignored.

The electrical parameters for the three physical regions are specified to correspond to best estimates for the conductivity and dielectric constant of the three regions. They are assumed fixed at all times. For study of parametric dependence, these parameters were changed from run to run.

For the ambient growth medium, and cell interior, the dielectric constant was set to 81, the value for water. Because the cell membrane effectively shields the interior from electric fields, the exact value for the interior region is not critical. In any event, it is likely that the electrical characteristics of the cell interior are dominated by the water in the parameter range of interest.

The value for the membrane parameters were obtained from previous work, with the relative dielectric constant being set to 6. The membrane is assumed to be insulating, so that a value for electrical conductivity of 10⁻⁷ Siemens/meter should be representative. The main point is that the membrane conductivity is many orders of magnitude lower than the ambient water.

Pulsed Field Study: The physical situation being modeled requires charge conservation, which means that charge can accumulate on surfaces at interfaces. As suggested in FIG. 3, this indicates a charge of different sign accumulating on the membrane surfaces. This has two consequences: (i) the charge generates very large electric fields within the membrane. For a typical cell size of 4 microns diameter, and a membrane thickness of 100 Angstroms, the peak electric field in the membrane is close to 3 MV/cm, which is 300 times higher than the far field and (ii) the charge interacts with the local electric field and generates forces on the membrane surfaces (inner and outer). This is represented formally by the Maxwell stress tensor. For normal purposes, this stress tensor in integrated over the upper hemispherical surface of the spherical cell to give a total force pulling the top half of the cell axially upwards or radially sideways (of course equal forces are acting on the lower hemisphere also).

To simulate typical experimental situations, a double exponential was used. Such a pulse is represented by an applied electric voltage of the form

V=V₀e^{-t/τ}^{₁}(1−e^{-t/τ}^{₂}). (1)

There are two time constants used here, with

τ₁=voltage decay time≈5μ seconds,

τ₂=voltage rise time≈0.5μ seconds. (2)

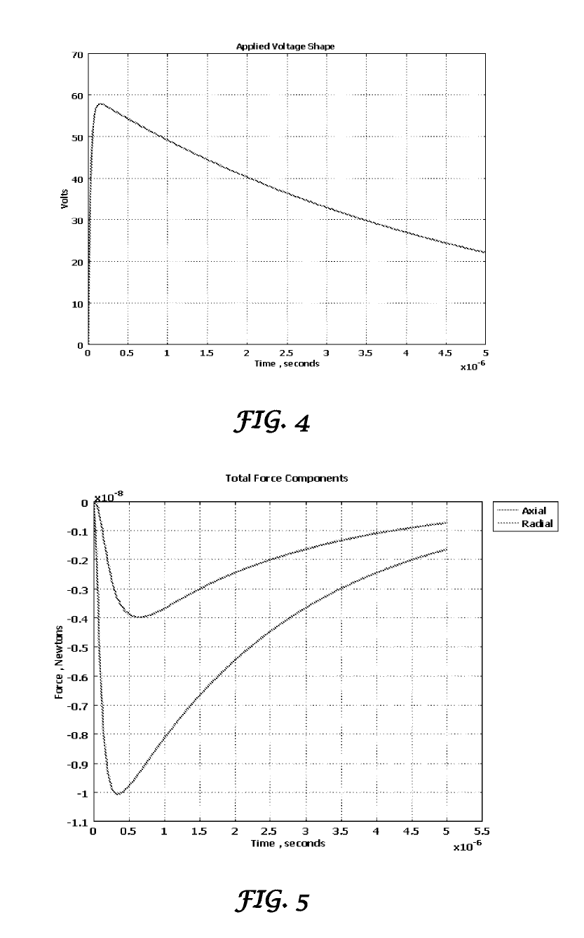

The voltage decay time is usually characterized by the time duration for which the voltage is greater than or equal to half its peak value—abbreviated as FWHM. This time is closely equal to 70% of the decay time constant. The pulse shape is shown in FIG. 4.

The value of water conductivity was set at 0.1 Siemens/meter to represent pond water. The numerical results for the membrane forces which are induced by this pulse are shown in

FIG. 5. Note that both the axial and radial forces are negative. It is also noted that the steady state results for force do not depend on the polarity of the applied voltage.

The meaning of the negative forces is that the resulting force directions are compressive, i.e., the forces want to squeeze the cell inward. Of most significance, the radial compression is the dominant component. The net result is that the cell membrane tends to be squeezed more in the radial direction. The cell then tends to elongate along the axis of the applied field, and is squeezed inward in the sideways direction. This is because the cell volume remains constant; as the dominant radial force squeezes in the cell, the axial length of the cell must increase to conserve volume.

The two forces are the integrated totals for all stresses acting on the top hemisphere. The actual stresses vary with position on the membrane. The axial stresses tend to peak at the top and bottom areas of the membrane, while the radial stresses tend to peak at the side areas of the membrane surface.

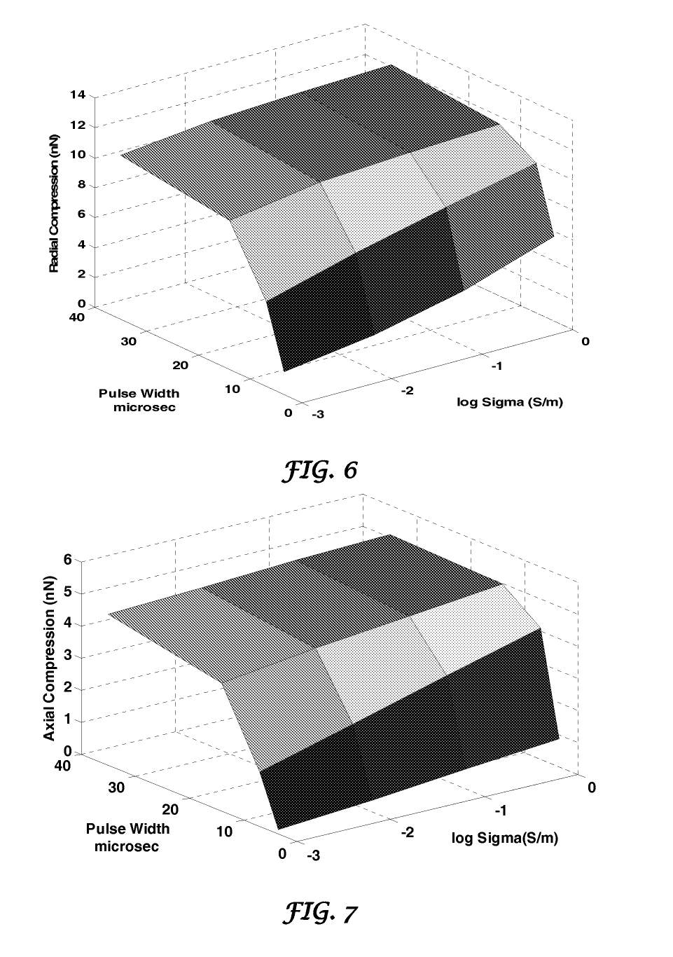

Simulations predicted how the peak forces generated depend on the duration of the applied electric field, full width at half maximum (FWHM), and the difference between electrical conductivity of the ambient growth medium and the intracellular material. The result for radial compression is shown in FIG. 6, while the corresponding axial compression force is shown in FIG. 7. The force values are in units of nanoNewtons (10⁻⁹ N), the exponential decay time constant is given as FWHM value in microseconds, and the water conductivity is characterized as the logarithm (base 10) of the conductivity in Siemens/meter.

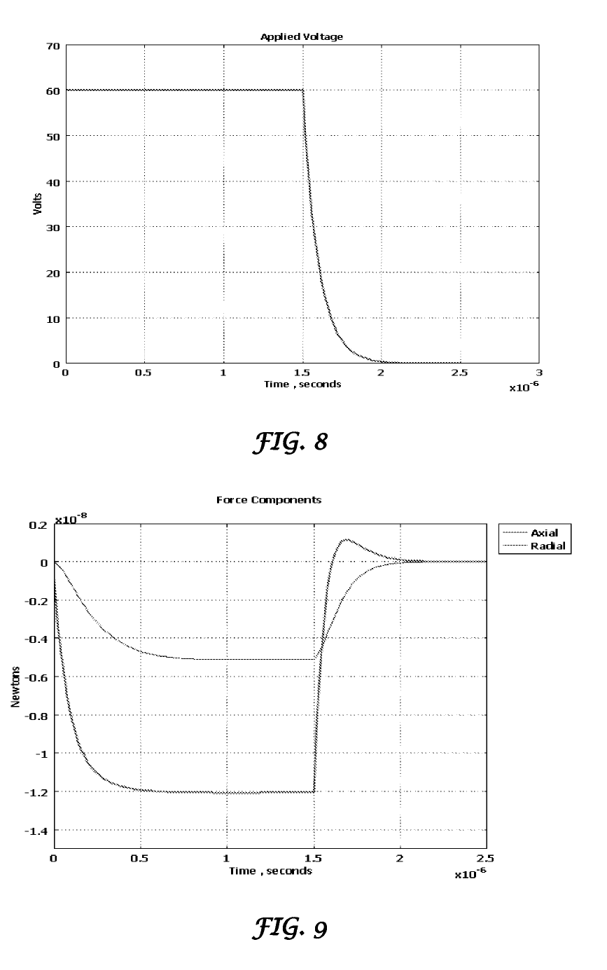

Force Reversal Study: Another interesting time dependent pulse shape has a constant amplitude voltage which is quickly (˜0.1 μs) switched off. This shape is shown in FIG. 8. The radial force acting on the cell membrane briefly reverses direction during the voltage turn-off. This can be seen in FIG. 9. This puts the cell membrane into a state of tension for a short time. This reversal results in lysing of the cell.

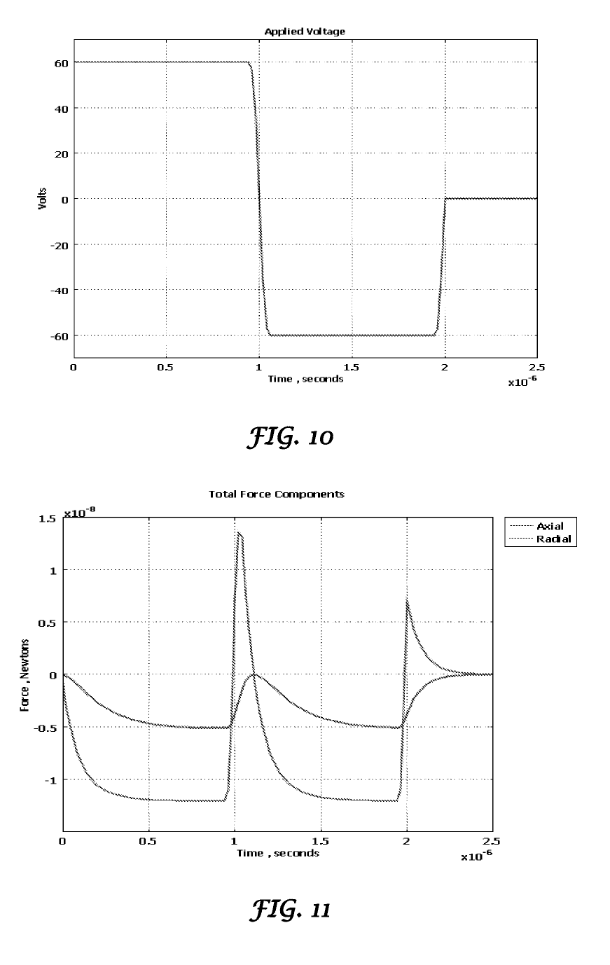

Simulations also showed voltage pulses which reverse polarity can be used to produce large force reversal. For this, a square wave type profile like that in FIG. 10 was used. For slow reversal of the voltage, no force reversal is observed. For more rapid voltage reversal, the distribution of induced surface charge does not have time to rearrange itself, and large force reversal is produced, as indicated in FIG. 11.

Measurements of Components of the Cytoplasm Released by the Electromechanical Lysis: Electromechanical lysis is a technique that ruptures algal cell walls through charge redistribution of the cell membranes. The result of applying varying pulses of voltage is cellular lysis and release of cytoplasmic components, including proteins and neutral lipids. Measurements of either or both of these provide an indication of the success of the lysing process. Proteins released into the incubating medium can readily be measured via the Bradford assay. This provides a method to verify lysis. To quantify neutral lipid release, a high-throughput method was developed using the neutral lipid fluorescent indicator BODIPY493/503 (Invitrogen), and the results were confirmed using the established Nile Red lipid indicator. Exposure to an appropriate electric field caused a significant increase in protein and neutral lipid release from Chlorella and Nannochloropsis, two relevant types of algae, over unpulsed controls. Furthermore, pulsing was as effective a lysing agent as applying high-sheer force (dounce), but at a fraction of the cost. A dounce homogenizer is generally accepted as a technique that produces nearly 100% lysing, so it was used as a reference for comparison.

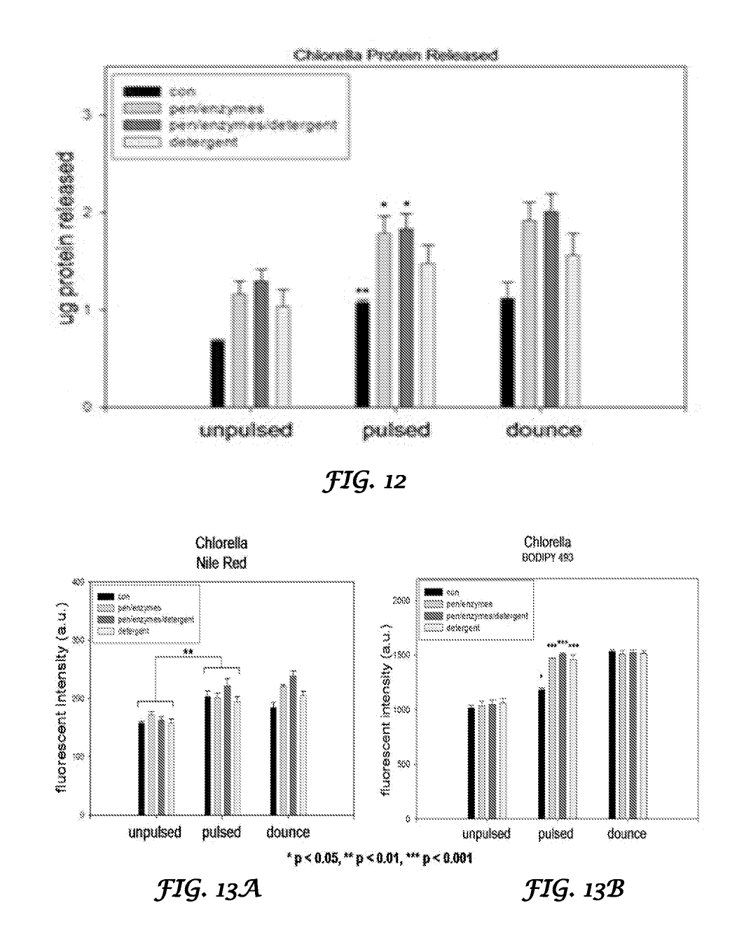

Analysis Data: FIG. 12 is a histogram showing protein release in Chlorella. In this figure, the negative control, i.e., unpulsed, is on the left, the pulsed sample is in the middle, and the positive control lysed using a dounce homogenizer is on the right. The protein release in the unpulsed samples was the lowest, while pulsed and the dounce homogenized samples produced nearly identical results.

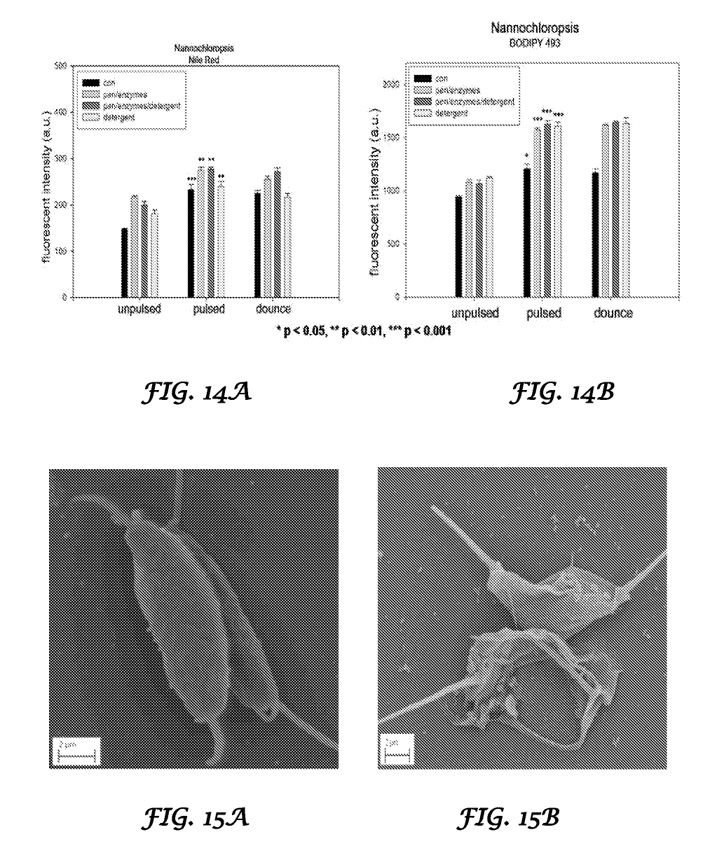

FIGS. 13A and 13B are histogram plots showing measured quantities of neutral lipid release. For Chlorella, a Nile red indicator (FIG. 13A) showed good agreement between the pulsed and the dounce treated samples. When soaps or other aids were used, both processes yielded the same results. Conducting the same study in Nannochloropsis (FIGS. 14A and 14B), as was conducted in Chlorella, yielded much the same results.

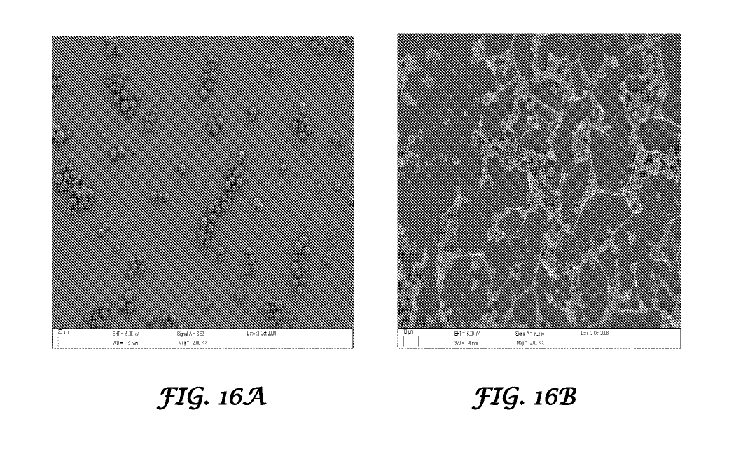

Visual Indicators of EM Lysis Effectiveness: In addition to the chemical measurements, lysing was verified using scanning electron microscopy. FIGS. 15A and 15B are scanning electron microscope photographs showing Scenedesmus cells before and after electromechanical lysing, respectively. The photographs show that the cells opened in response to the electrically induced mechanical force. The failure is obvious, producing a significant opening. FIGS. 16A and 16B are scanning electron microscope photographs of samples of different types of failure. Here, the more spherical algae Chlorella appears to have failed by collapsing and squeezing out the cytoplasm. The different failure modes between the Scenedesmus and the Chlorella are presumably due to different mechanical properties in different algae types.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

United States Patent Publication No. 20080220491: Method and Device for Electroporation of Biological Cells.

U.S. Patent Publication No. 20090061504: Apparatus for Performing Magnetic Electroporation.

U.S. Patent Publication No. 20090087900: Apparatus for Performing Electrodistention on Algae Cells.

What is claimed is:

1. A method for electrical treatment of one or more biological cells comprising the steps of:
providing the one or more biological cells suspended or surrounded by a lysing medium comprising a fresh water, a salt water, a brackish water, a growth medium, a culture medium or combinations thereof, wherein an electrical conductivity of the lysing medium is different from the electrical conductivity of a cell membrane and the cytoplasm of the one or more biological cells;
applying a time varying electromagnetic field to the one or more biological cells using one or more electrode pairs placed within or externally to the lysing medium, wherein the electromagnetic field applies a mechanical force on a cell membrane comprising a force stress; and
applying and rapidly switching off one or more voltage pulses to the one or more biological cells resulting in lysis of the one or more biological cells.

2. The method of claim 1, further comprising the steps of:
releasing one or more cellular components from the lysed biological cells into the lysing medium; and
separating and collecting the released cellular components for further processing.

3. The method of claim 2, wherein the cellular components comprise neutral lipids, proteins, triglycerides, sugars or combinations and modifications thereof.

4. The method of claim 3, wherein the neutral lipids, triglycerides or both are converted to yield a fatty acid methyl ester (FAME), a biodiesel or a biofuel.

5. The method of claim 1, wherein the one or more biological cells comprise algal cells, bacterial cells, viral cells or combinations thereof

6. The method of claim 5, wherein the algal cells are selected from a division comprising Chlorophyta, Cyanophyta (Cyanobacteria), Rhodophyta (red algae), and Heterokontophyt.

7. The method of claim 5, wherein the one or more algal cells comprise microalgae selected from a class comprising Bacillariophyceae, Eustigmatophyceae, and Chrysophyceae.

8. The method of claim 7, wherein the microalgal genera are selected from the group consisting of Nannochloropsis, Chlorella, Dunaliella, Scenedesmus, Selenastrum, Oscillatoria, Phormidium, Spirulina, Amphora, and Ochromonas.

9. The method of claim 7, wherein the microalgal species are selected from the group consisting of Achnanthes orientalis, Agmenellum spp., Amphiprora hyaline, Amphoracoffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorellakessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Effipsoidon sp., Euglena spp., Franceia sp., Fragilaria crotonensis, Fragilaria sp., Gleocapsa sp., Gloeothamnion sp., Haematococcus pluvialis, Hymenomonas sp., lsochrysis aff. galbana, lsochrysis galbana, Lepocinclis, Micractinium, Micractinium, Monoraphidium minutum, Monoraphidium sp., Nannochloris sp., Nannochloropsissalina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrine, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheriaacidophila, Pavlova sp., Phaeodactylum tricomutum, Phagus, Phormidium, Platymonas sp., Pleurochrysis camerae, Pleurochrysis dentate, Pleurochrysis sp., Prototheca wickerhamii, Prototheca stagnora, Prototheca portoricensis,Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

10. The method of claim 1, the electrical treatment is carried out in a batch or a continuous processing mode.

11. The method of claim 1, wherein a strength of the applied electromagnetic field ranges from 0.5 kV/cm to 500 kV/cm.

12. The method of claim 1, wherein the electromagnetic field is applied for a time duration ranging from a tenth of a microsecond to a few tens of microseconds.

13. An electromechanical lysing method for releasing one or more cellular components of from one or more algal cell membranes comprising the steps of:
providing one or more algal cells suspended or surrounded by a lysing medium comprising a fresh water, a salt water, a brackish water, a growth medium, a culture medium or combinations thereof, wherein an electrical conductivity of the lysing medium is different from the electrical conductivity of the cell membrane and of a cytoplasm of the one or more algal cells, wherein the algal cells comprise flocculated or uflocculated algal cell cultures;
applying a time varying electromagnetic field to the algal cells using one or more electrode pairs placed within or external to the lysing medium, wherein the electromagnetic field applies a mechanical force on the algal cell membrane comprising a force stress;
applying and rapidly switching off one or more voltage pulses to the one or more algal cells resulting in a lysis of the algal cells; and
lysing the one or more algal cells to release one or more cellular components into the lysing medium.

14. The method of claim 13, wherein the cellular components comprise neutral lipids, proteins, triglycerides, sugars or combinations and modifications thereof by electroporation

15. The method of claim 13, further comprising the steps of:
separating and collecting the neutral lipids, the triglycerides or both from the released cellular components for further processing; and
converting the neutral lipids, the triglycerides or both to yield a fatty acid methyl ester (FAME), a biodiesel or a biofuel.

16. The method of claim 13, wherein the algal cells comprise microalgae or macroalgae selected from the group consisting of diatoms (bacillariophytes), green algae (chlorophytes), blue-green algae (cyanophytes), golden-brown algae (chrysophytes), haptophytes, freshwater algae, saltwater algae, Amphipleura, Amphora, Chaetoceros, Cyclotella, Cymbella, Fragilaria, Hantzschia, Navicula, Nitzschia, Phaeodactylum, Thalassiosira Ankistrodesmus, Botryococcus, Chlorella, Chlorococcum, Dunaliella, Monoraphidium, Oocystis, Scenedesmus, Nannochloropsis, Tetraselmis, Chlorella, Dunaliella, Oscillatoria, Synechococcus, Boekelovia, Isochysis, and Pleurochysis.

17. The method of claim 13, wherein the algae is Chlorella or Nannochloropsis.

18. The method of claim 13, wherein a cell density of the one or more algal cells ranges from a single cell to a largest cell density, wherein an external electrical conductivity is determined by the lysing medium

19. The method of claim 13, wherein the strength of the applied electromagnetic field ranges from 0.5 kV/cm to 500 kV/cm.

20. The method of claim 13, wherein the electromagnetic field is applied for a time duration ranging from a tenth of a microsecond to a few tens of microseconds.

21. The method of claim 13, the lysing is carried out in a batch or a continuous processing mode.

22. A method for lysing a flocculated or unflocculated algal cell culture to release one or more cellular components comprising neutral lipids, proteins, triglycerides, sugars or combinations and modifications thereof by electroporation of an algal cell membrane comprising the steps of:
providing the one or more flocculated or unflocculated algal cell cultures suspended or surrounded by a lysing medium comprising a fresh water, a salt water, a brackish water, a growth medium, a culture medium or combinations thereof, wherein an electrical conductivity of the lysing medium is different from the electrical conductivity of the cell membrane of the one or more algal cells;
applying multiple pulses of a time varying electromagnetic field to the flocculated or unflocculated algal cells using one or more electrode pairs placed within or external to the lysing medium, wherein the electromagnetic field applies a mechanical force comprising a radial force stress compressing the cells inward along a radial direction of the applied electromagnetic field and an axial force stress elongating the cells in a direction along an axis of the applied electromagnetic field;
applying and rapidly switching off one or more voltage pulses to the flocculated or unflocculated algal cells;
inducing a reversal in the direction of the radial force stress followed by an expansion of the cells in the radial direction causing a lysis of the algal cells; and
lysing the one or more algal cells to release one or more cellular components into the lysing medium.

23. The method of claim 22, further comprising the steps of:
separating and collecting the neutral lipids, the triglycerides or both from the released cellular components for further processing; and
converting the neutral lipids, the triglycerides or both to yield a fatty acid methyl ester (FAME), a biodiesel or a biofuel.

24. The method of claim 22, wherein the algal cells comprise microalgae or macroalgae selected from the group consisting of diatoms (bacillariophytes), green algae (chlorophytes), blue-green algae (cyanophytes), golden-brown algae (chrysophytes), haptophytes, freshwater algae, saltwater algae, Amphipleura, Amphora, Chaetoceros, Cyclotella, Cymbella, Fragilaria, Hantzschia, Navicula, Nitzschia, Phaeodactylum, Thalassiosira Ankistrodesmus, Botryococcus, Chlorella, Chlorococcum, Dunaliella, Monoraphidium, Oocystis, Scenedesmus, Nannochloropsis, Tetraselmis, Chlorella, Dunaliella, Oscillatoria, Synechococcus, Boekelovia, Isochysis, and Pleurochysis.

25. A system for producing a biodiesel, a fatty acid methyl ester (FAME), a biofuel or combinations and modifications thereof from an algal cell culture comprising:
an algal growth tank or a cultivation tank for growing the one or more algal species in a presence of water and other growth factors selected from the group consisting of nutrients, minerals, CO₂, air, and light;
a harvesting vessel for harvesting the cultivated algae from the growth tank, wherein the algae are harvested by one or more methods selected from the group consisting of centrifugation, autoflocculation, chemical flocculation, froth flotation, and ultrasound;
a concentration tank wherein the harvested algae is dewatered to concentrate the algae;
a lysis tank or a chamber comprising a lysing medium for electromechanically lysing the concentrated algae to release one or more cellular components comprising neutral lipids, proteins, triglycerides, sugars or combinations and modifications thereof, wherein an electrical conductivity of the lysing medium is different from the electrical conductivity of an algal cell membrane and cytoplasm, wherein the lysing is accomplished by a device comprising:
single or multiple pairs of electrodes for applying a single pulse or multiple pulses of a time varying electromagnetic field to the algal cells, wherein the electromagnetic field applies a mechanical force on the algal cell membrane; and
an apparatus for applying and rapidly switching off one or more voltage pulses to the algal cells resulting in a reversal in the direction of the radial force stress to induce an expansion of the cells in the radial direction causing a lysis of the algal cells;
a separation vessel for separating the released algal lipids and triglycerides from the lysing medium and other released cellular components; and
a reaction vessel for converting the separated algal lipids, triglycerides to a biodiesel, a FAME, a biofuel or combinations or modifications thereof by a transesterification reaction.

26. The system of claim 25, wherein the algal species comprise microalgae or macroalgae selected from the group consisting of diatoms (bacillariophytes), green algae (chlorophytes), blue-green algae (cyanophytes), golden-brown algae (chrysophytes), haptophytes, freshwater algae, saltwater algae, Amphipleura, Amphora, Chaetoceros, Cyclotella, Cymbella, Fragilaria, Hantzschia, Navicula, Nitzschia, Phaeodactylum, Thalassiosira Ankistrodesmus, Botryococcus, Chlorella, Chlorococcum, Dunaliella, Monoraphidium, Oocystis, Scenedesmus, Nannochloropsis, Tetraselmis, Chlorella, Dunaliella, Oscillatoria, Synechococcus, Boekelovia, Isochysis, and Pleurochysis.

27. A device for electromechanical treatment of one or more biological cells comprising:
a chamber or a vessel comprising flocculated or unflocculated biological cells suspended or surrounded by a lysing medium comprising fresh water, salt water, brackish water, a growth medium, a culture medium or combinations thereof, wherein an electrical conductivity of the lysing medium is different from the electrical conductivity of a cell membrane of the one or more biological cells;
one or more pairs of electrodes for applying single or multiple pulses of a time varying electromagnetic field to the biological cells, wherein the one or more pairs of electrodes are placed within or external to the chamber, wherein the electromagnetic field applies a mechanical force on the cell membrane comprising a radial force stress compressing the cells inward along a radial direction of the applied electromagnetic field and an axial force stress elongating the cells in a direction along an axis of the applied electromagnetic field;
an apparatus for applying and rapidly switching off one or more constant amplitude voltage pulses to the biological cells resulting in a reversal in the direction of the radial force stress followed by an expansion of the cells in the radial direction causing a lysis of the algal cells; and
one or more optional collecting vessels, receivers, separators or combinations for processing the released cellular components.

28. The device of claim 27, wherein the electrodes are profiled to create an uniform field and minimal voltage stress concentration.

29. The device of claim 27, wherein the cellular components comprise neutral lipids, proteins, triglycerides, sugars or combinations and modifications thereof

30. The device of claim 27, wherein the neutral lipids, triglycerides or both are converted to yield a fatty acid methyl ester (FAME), a biodiesel or a biofuel.

31. The device of claim 27, wherein the one or more biological cells comprise algal cells, bacterial cells, viral cells or combinations thereof.

32. The device of claim 31, wherein the algal cells are selected from a division comprising Chlorophyta, Cyanophyta (Cyanobacteria), Rhodophyta (red algae), and Heterokontophyt.

33. The device of claim 31, wherein the one or more algal cells comprise microalgae selected from a class comprising Bacillariophyceae, Eustigmatophyceae, and Chrysophyceae.

34. The device of claim 33, wherein the microalgal genera are selected from the group consisting of Nannochloropsis, Chlorella, Dunaliella, Scenedesmus, Selenastrum, Oscillatoria, Phormidium, Spirulina, Amphora, and Ochromonas.

35. The device of claim 33, wherein the microalgal species are selected from the group consisting of Achnanthes orientalis, Agmenellum spp., Amphiprora hyaline, Amphoracoffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorellakessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Effipsoidon sp., Euglena spp., Franceia sp., Fragilaria crotonensis, Fragilaria sp., Gleocapsa sp., Gloeothamnion sp., Haematococcus pluvialis, Hymenomonas sp., lsochrysis aff galbana, lsochrysis galbana, Lepocinclis, Micractinium, Micractinium, Monoraphidium minutum, Monoraphidium sp., Nannochloris sp., Nannochloropsissalina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrina, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheriaacidophila, Pavlova sp., Phaeodactylum tricomutum, Phagus, Phormidium, Platymonas sp., Pleurochrysis camerae, Pleurochrysis dentate, Pleurochrysis sp., Prototheca wickerhamii, Prototheca stagnora, Prototheca portoricensis,Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

36. The device of claim 27, the electrical treatment is carried out in a batch or a continuous processing mode.

37. The device of claim 27, wherein the strength of the applied electromagnetic field ranges from 0.5 kV/cm to 500 kV/cm.

38. The device of claim 27, wherein the electromagnetic field is applied for a time duration ranging from a tenth of a microsecond to a few tens of microseconds.

39. A device for electrical treatment for a release of one or more cellular components comprising neutral lipids, proteins, triglycerides, sugars or combinations and modifications thereof from one or more flocculated or unflocculated algal cell cultures comprising:
a chamber or a vessel comprising flocculated or unflocculated algal cells suspended or surrounded by a lysing medium comprising fresh water, salt water, brackish water, a growth medium, a culture medium or combinations thereof, wherein an electrical conductivity of the lysing medium is different from the electrical conductivity of a cell membrane and intracellular material of the one or more algal cells;
one or more pairs of electrodes for applying single or multiple pulses of a time varying electromagnetic field to the algal cells, wherein the electromagnetic field applies a mechanical force on the cell membrane;
an apparatus for applying and rapidly switching off one or more voltage pulses to the algal cells resulting in a reversal in the direction of the radial force stress followed by an expansion of the cells in the radial direction causing a lysis of the algal cells; and
one or more optional collecting vessels, receivers, separators or combinations for processing the released cellular components.

40. The device of claim 39, wherein the neutral lipids, the triglycerides or both are converted to yield a fatty acid methyl ester (FAME), a biodiesel or a biofuel.

41. The device of claim 39, wherein the algal cells comprise microalgae or macroalgae selected from the group consisting of diatoms (bacillariophytes), green algae (chlorophytes), blue-green algae (cyanophytes), golden-brown algae (chrysophytes), haptophytes, freshwater algae, saltwater algae, Amphipleura, Amphora, Chaetoceros, Cyclotella, Cymbella, Fragilaria, Hantzschia, Navicula, Nitzschia, Phaeodactylum, Thalassiosira Ankistrodesmus, Botryococcus, Chlorella, Chlorococcum, Dunaliella, Monoraphidium, Oocystis, Scenedesmus, Nannochloropsis, Tetraselmis, Chlorella, Dunaliella, Oscillatoria, Synechococcus, Boekelovia, Isochysis, and Pleurochysis.

42. The device of claim 39, wherein the algae is Chlorella or Nannochloropsis.
